(19) United States
(12) Patent Application Publication
(10) Pub. No.: US 2005/0228240 A1
(43) Pub. Date: Oct. 13, 2005

(54) Devices and methods for characterizing nervous system impairment

(75) Inventors: Petra Williams, Columbus, OH (US); Deborah S. Nichols-Larsen, Dublin, OH (US); D. Michele Basso, Columbus, OH (US)

Correspondence Address: CALFEE HALTER & GRISWOLD, LLP, 800 SUPERIOR AVENUE, SUITE 1400, CLEVELAND, OH, 44114, US

(73) Assignee: The Ohio State University, Columbus, OH (US)

(21) Appl. No.: 10/957,780

(22) Filed: Oct. 4, 2004

Related U.S. Application Data
(60) Provisional application No. 60/508,728, filed on Oct. 3, 2003.

(51) Int. Cl. A61B 5/00

(52) U.S. Cl. 600/300

(57) ABSTRACT

Devices and methods are provided for assessing and characterizing the degree of nervous system impairment in subjects who have sustained injury to the nervous system. The devices and methods involve use of test objects with physical properties that can be discerned by prehension and tactile sensation, and that directly influence the hand forces that are used to manipulate the test objects.

### PRIORITY CLAIM

This application claims priority to U.S. Provisional Patent Application 60/508,728, filed Oct. 3, 2003, which is incorporated herein by reference, in its entirety.

### BACKGROUND

Impairment to the nervous system caused by stroke or other injury can be associated with a wide and variable range of functional loss. Optimal treatment and rehabilitation of affected individuals depends at least in part on the accurate assessment of the nature and degree of the injury and the resultant impairment. Two factors that influence designing an effective rehabilitation plan include identification of the locus of injuries in the brain, and objective measurement of the extent of impairment. Stroke survivors and other individuals with nervous system injury often experience motor and somatosensory impairments which interfere with their ability to actively explore objects for sensory information. Several methods have been developed to characterize nervous system impairment by assessing somatosensory acuity or motor performance. These various methods provide some information to aid in defining the extent of impairment, however, they are of limited value because they involve subjective characterization of a subject's status, and do not provide an independent index for quantifying the degree of impairment. Accordingly, a new method of evaluation is required that can provide reliable and independent measurement of degree of impairment.

### SUMMARY

Devices and methods are provided for measuring sensory ability in order to characterize the degree of impairment in individuals, specifically human subjects, who have sustained injury to the nervous system. In some embodiments, devices for testing sensory ability comprise a test set having an even number of test objects which are essentially identical (do not vary by more than 20%) in either size or shape, or both. In some embodiments, the test objects are essentially identical in both size and shape. In other embodiments, the test objects may all be of the same size, but each may have a different shape. In yet other embodiments, the test objects may be of different sizes, but all have the same shape. The test set comprises two essentially identical subsets, wherein each subset has either an even or an odd number of test objects. Each of the test objects in a subset varies in one or both of a first and a second parameter. The devices may comprise 16, 18, 20, 22, 24, 26, 28, 30, or more test objects, in even numbers; thus, subsets may comprise 8, 9, 10, 11, 12, 13, 14, 15, or more test objects, wherein additional test objects may be desirable with respect to the statistical significance of the results obtained with the test. Good results have been obtained with test sets having 18 objects, with 9 test objects in each of two subsets.

The variations in the first and second parameters can be assessed by grasping or holding the test object and exploring its surface, ie., prehension and tactile sensation These varied parameters directly influence the hand forces used to manipulate the test objects. It is desirable that the varied parameters can not be readily assessed by visual or other analysis, thus avoiding the risk that the subject will be able to use skills other than prehension and tactile sensation to discern variations. The varied parameters may be detected in some or all of the test objects by visual inspection. Alternatively, the varied parameters in some or all of the test objects may not be visualized, and may be detected only upon grasping the test objects.

At least two or more test objects in a subset share the same increment of at least one parameter. In some embodiments, three test objects in a subset share the same increment of at least one parameter. For example, in a test set having 18 test objects in which there are three different increments for each parameter, there will be three test objects in each subset that share the same increment for each parameter, however, no two test objects in a subset share the same increment of both the first and the second parameter. And since there are two identical subsets, exactly two test objects in the test set will share the same increment of both the first and the second parameter.

Test objects have shapes that have a regular or uniform surface shape that is free of protrusions, voids and edges, so as to minimize distraction and limit the requirement for the test subject to “learn” the best way to grip the test object. In some embodiments, test objects have shapes which are common or familiar and are routinely encountered in every day life. According to such embodiments, each of the test objects in the test set is three-dimensional in shape and is of sufficient size to fit within and substantially occupy the grasp of a human hand.

The varied parameters can be discerned by prehension and tactile sensation and directly influence the hand forces that are used to manipulate a test object (i.e, influence handling so as to avoid dropping or crushing the test object). In some embodiments, the parameters of weight and texture of test objects are varied. According to such embodiments, both the weight and surface textures of each test object varies from the others such that each test object in a subset has a different combination of weight and surface texture. Further according to such embodiments, no more than two test objects in the test set share both the same weight and the same surface texture, thus, no two test objects in a subset share both the same weight and the same surface texture. Test objects may be provided in a range of size categories, such as small, medium and large, so as to accommodate the broadest range of hand sizes, as may be found in pediatric subjects at the one extreme, and very large adult subjects at the other extreme.

The invention also provides methods for testing sensory ability comprise providing a device according to the present disclosure which is used in the assessment of sensory ability and testing a subject's ability to match target objects using a match to sample design. More particularly, the method involves using a testing device as disclosed herein in a test setting involving a text examiner and a test subject, wherein both the examiner and the subject are seated at opposite sides of a table, with a screen or curtain disposed between the two so as to prevent visual contact. Test objects from a test set of a device disclosed herein are arranged in front of the examiner and on the opposite side of the barrier from the test subject, but reachable by the test subject. The subject is presented with various combinations of test objects wherein one object is a “target object” and the remaining objects are different from one another but one matches the target object. The test objects are labeled on the test subject's side of the screen as “T, 1, 2, 3, (etc.).” The test subject is instructed to actively explore the target object by grasping, and then to evaluate each of the test objects to find the match. The test subject is evaluated based on the time taken to propose a match and the accuracy of the proposal. The test is then repeated for additional trials using other combinations of each of the test objects in the test set until all such possible combinations have been exhausted. The test subject's overall accuracy score is then calculated by adding together the number of correct matches. The test subject's average time to match score is calculated by adding all of the recorded times and dividing by the number of trials.

Use of the various embodiments of devices and methods enables the assessment of nervous system impairment of subjects. The results of such assessment may be compared between subjects and may also be used to measure the progress of a subject over a period of time, such as through a course of therapy.

### BRIEF DESCRIPTION OF THE DRAWINGS

FIG. 1 shows one embodiment of a subset of test objects; panel A shows a full subset of nine test objects with a common object (small juice can) after which the test objects were modeled; panels B and C show combinations of four test objects within a set arranged as they would be encountered by a subject.

FIG. 2 shows the Active Sensation Test as administered to a subject.

FIG. 3 shows an embodiment of a sample data collection form for use in connection with the Active Sensation Test.

FIG. 4 shows a graphical presentation of the sensitivity and specificity data in a Receiver Operating Characteristic (ROC) Curve.

FIG. 5 shows Active Sensation Test overall accuracy scores for stroke survivor and control subjects in the form of a Receiver Operating Characteristic (ROC) curve.

FIG. 6 shows Active Sensation Test average time to match scores for stroke survivor and control subjects in 15 second increments.

FIG. 7 shows change score distribution for W&T Test accuracy score

### DETAILED DESCRIPTION

The present invention will now be described with occasional reference to the specific embodiments of the invention. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to that this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise. All publications, patent applications, patents, and other references mentioned herein are incorporated by reference in their entirety.

Unless otherwise indicated, all numbers expressing quantities, properties such as weight, and so forth, as used in the specification and claims are to be understood as being modified in all instances by the term “about.” Accordingly, unless otherwise indicated, the numerical properties set forth in the following specification and claims are approximations that may vary depending on the desired properties sought to be obtained in embodiments of the present invention. Notwithstanding that the numerical ranges and parameters setting forth the broad scope of the invention are approximations, the numerical values set forth in the specific examples are reported as precisely as possible. Any numerical values, however, inherently contain certain errors necessarily resulting from error found in their respective measurements.

The disclosure of all patents, patent applications (and any patents that issue thereon, as well as any corresponding published foreign patent applications), and publications that may be mentioned throughout this description are hereby incorporated by reference herein. It is expressly not admitted, however, that any of the documents incorporated by reference herein teach or disclose the present invention.

The present invention may be understood more readily by reference to the following detailed description of the embodiments of the invention and the Examples included herein. However, before the present methods, compounds and compositions are disclosed and described, it is to be understood that this invention is not limited to specific methods, specific devices, specific materials, or specific conditions, etc., as such may, of course, vary, and the numerous modifications and variations therein will be apparent to those skilled in the art. It is also to be understood that the terminology used herein is for the purpose of describing specific embodiments only, and is not intended to be limiting.

Sensory Tests for Evaluating Nervous System Impairment

The principal functional use of the upper extremities involves reaching for and interacting with objects in the environment through prehension (grasping in the hand). Both sensory and motor abilities are required for successful prehension. Prehension can be used to explore an object in order to gather information about the object such as the weight of the hammer and the texture of the hammer handle nail—this aspect of prehension involves sensory skills. Simultaneously, prehension can be used to perform a physical task with an object such as using hammer to hit a nail—this aspect of prehension involves motor skills.

Sensation plays an important role in the ability of individuals to reach, grasp, and manipulate objects with the hand. Thus, measurement of hand sensation is an element in evaluating arm and hand function, and is part of a process of sensory assessment by clinicians assessing and treating patients with nervous system impairment. Some sensory tests available in the art are designed to evaluate isolated sensory modalities including touch/pressure, position sense, pain, and temperature. Examples of these sensory tests include: the Semmes-Winstein monofilaments, which measures the minimum force that can be sensed; the Disk-Criminator, which measures the minimum distance between two points that can be perceived as two distinct points of pressure; and the Wrist Position Sense Test, which measures the accuracy of position sense at the wrist joint. Other sensory tests available in the art are functional tests designed to assess the ability of the subject to use an affected hand by drawing upon whatever sensation is available. Examples of these functional tests include: identifying an object (such as a key, or a button) from touch (stereognosis); and a variety of dexterity tests, such as timed peg placement tests or pick-up (sticks) tests. None of these tests allows assessment of a test subject's ability to sense and discriminate between test objects based on active exploration of the test objects' objective and inherent properties.

Active Sensation Test (“AST”)

According to the devices and methods disclosed herein, the Active Sensation Test permits evaluation of the ability of the entire hand of a test subject to gather information about varied properties of test objects using prehension and tactile input. The information extracted from this active exploration generates diverse perceptions called object properties. Object properties refer to characteristics or dimensions of an object including its size, shape, weight, texture, surface compliance, and temperature. The AST permits measure of hand function based on how object property information is gathered and used to perform manual tasks during daily life.

Comparison of AST with Other Tests:

The Active Sensation Test is a unique test of sensory ability differing from other sensory tests not only by what is measured but also by the method of measuring.

First, the AST test design—test object construction and match-to-sample trials—bypasses the innate challenges to reliability inherent to most other sensory tests available. The primary threat to reliability—and even to validity—in sensory testing is inconsistent stimulus application. In isolated sensory modality testing the method demands that the examiner reproduce the same magnitude of stimulus every time—e.g. use the same pressure and velocity when applying a poke. These methods not only make the testing dependent upon examiner skill but also make it nearly impossible to definitively evaluate the effects of an intervention or recovery. The AST test object construction offers the potential for precise stimulus control independent of examiner skill.

Second, the AST score provides an objective and quantitative measure of hand function as it relates to the ability explore in order to feel a test object. Other sensory tests provide a threshold measure, or some minimum quality of a sensation that the subject can report as perceived. The AST match-to-sample design eliminates examiner subjectivity and is independent of examiner opinion. In contrast to other testing methods, the objective score produced by the AST permits accurate comparisons of subject performance over time in order to evaluate progress of a single subject and to distinguish subjects from each other. For example, in stereognosis based tests, subjects may be scored by the number of objects correctly identified or by the number identified in a particular period of time; the significance of knowing a “pencil” and not being able to identify a “key” or vice versa has not been established. Likewise, the dexterity tests are timed as well with a score of how many pegs are placed in a particular period of time, but these tests do provide data that can measure improvement and be used to compare between subjects.

The AST differs from the isolated sensory modality tests in the following ways: The AST evaluates haptic touch or the integration of all the sensory modalities found in the hand—touch/pressure, position sense, and temperature. The various isolated sensory modality tests evaluate a single component of sensation. During the AST, the hand is actively moving or touching/feeling the object. In other words, the hand purposefully pursues sensory information about the object. The isolated sensory modalities are assessed by passively applying a stimulus to the skin (poke or prick) or to a joint (bend or straighten). The AST provides more relevant information, as most of everyday sensory experience is haptic touch—bjects are described by their properties as opposed to a collection of isolated component sensations.

The AST differs from the functional hand tests in the following ways: Even though the functional hand tests require active hand movements of objects, successful AST completion does not require object property identification. The goal of the AST is to find an object that feels the same as the target object. In stereognosis tests, subjects are required to identify an object by name e.g. “key”. This adds an extra layer of cognition and language that may complicate the test results. Dexterity tests, while once again active, evaluate prehension in the context of accomplishing a physical task e.g. place a small peg in a hole. The AST evaluates prehension in the context of perception.

AST Test Objects:

AST Test objects are three dimensional, and of a size and shape configuration that can be easily and reliable replicated so that they are essentially identical in size and shape, that is, the objects do not vary from one another in either size, or in shape, by more than 1, 2, 3, 4, 5, 6, 7, 8, 9, 10, 11, 12, 13, 14, 15, 16, 17, 18, 19, or 20%. Test objects are configured so as to be readily and easily grasped in one and held so as to substantially and comfortably fill the grip of human hands of varying sizes, and are of a size and shape that is suited to be held with the entire palmar surface of the hand rather than between the thumb and fingers (pincer grasp). Test objects are intended to be discriminated by a test subject based on properties that can be best distinguished by active exploration (i.e., grasping, lifting, gripping and rotating). Test object identification is not sought from the test subject, and test objects are designed to have minimal structural features so as to minimize the focus of the test user's attention on attempting to identify a test object.

Test objects may be provided in a range of size categories, such as small, medium and large, so as to accommodate the broadest range of hand sizes, as may be found in pediatric subjects at the one extreme, and very large subjects at the other extreme. Test objects have shapes which are common or familiar and are routinely encountered in every day life, and are designed to have shapes that have a regular or uniform surface shape that is free of protrusions, voids, and edges, so as to minimize distraction and limit the requirement for the test subject to “learn” the best way to grip the test object. For example, test objects may be cylindrical like a small juice can, spherical like a ball, or ovoid like an egg. In one embodiment, the size, shape and dimensions of the objects may be similar to the dimensions of 5.5 ounce juice can—they are cylindrical, and may be made, for example, from 1½ inch interior diameter PVC pipe, with 1 ⅞ inch diameter and 3⅞ inch height.

The object properties of test objects are varied in at least two key features that can be discerned by prehension and tactile sensation and are not influenced or biased by other means of analysis by a subject. The key features directly influence the hand forces that are used to manipulate a test object (i.e, influence handling so as to avoid dropping or crushing the test object). In some embodiments, the weight and texture of test objects are varied. Sets of test objects comprise two essentially identical subsets, such that a set has an even number of test objects and each subset has either an even or an odd number of test objects. The at least two key features are co varied in a substantially identical fashion within each subset. For example, if the key features of weight and surface texture are varied, then within a subset of test objects, no two test objects will have both the same weight and the same surface texture. Likewise, if temperature and surface texture are varied, then within a subset of test objects, no tow object will have both the same temperature and the same surface texture. Subset size is dependent on the number of variations in each of the varied key features. For example, there may be three different weights and three different textures in a subset. By co-varying these two key features, there would be a total of nine different test objects in a subset; thus, there would be a total of eighteen test objects in a set.

Parameters are varied by increments. For example, where the parameter of weight is varied, increments are different weights, such as described in the examples set forth herein. When the parameter of texture is varied, the increments may be in the form of different coarseness of texture, or stickiness, smoothness, or other superficial textural qualities.

Active Sensation Test Devices

An AST device comprises a set of test objects which includes two identical subsets, one subset serving as target objects, and one subset serving as match objects when used according to the methods described herein. According to one embodiment, an AST device comprises a set having a total of 18 test objects divided into 2 subsets of 9 test objects. One subset serves as target objects and the other subset as the match objects. Each set of test objects consists of 6 different groups of 3 test objects. As depicted in Table 1, a group has either the same weight with different textures (columns in Table 1) or the same texture with different weights (rows in Table 1). For example, while the test objects in group 1 have the same texture of paper, they do not weigh the same. Conversely, the test objects in group 4 weigh the same, 6 ounces, but have different textures. Each test object is a member of 2 groups—object number 1 is in group 1 and in group 4.

Using a 5.5 ounce juice can as model for test objects according to one embodiment, Table 1 profiles a possible set of 18 test objects, (grouped based on their textures and weights. Three textures crossed with three weights create 9 distinct objects. The columns in the table show three groups of objects that have the same texture per group but different weights per object (e.g. objects 1, 2, 3). The rows in the table show three groups of objects that have the same weight per group, but different textures per object (e.g. objects 1, 4, 7).

TABLE 1

| | Group 1 | Group 2 | Group 3 |
|---|---|---|---|
| | Texture 1 | Texture 2 | Texture 3 |
| | Paper | Cork | Plastic |
| Group 4 | #1 | #4 | #7 |
| Weight 1 | paper × 6 oz | cork × 6 oz | plastic × 6 oz |
| 6 ounces | | | |
| Group 5 | #2 | #5 | #8 |
| Weight 2 | paper × 7 oz | cork × 7 oz | plastic × 7 oz |
| 7 ounces | | | |
| Group 6 | #3 | #6 | #9 |
| Weight 3 | paper × 8 oz | cork × 8 oz | plastic × 8 oz |
| 8 ounces | | | |

According to the described embodiment of test device, the features that vary between test objects in the set are texture and weight; all other object features are essentially identical. In other words, the objects are the same size, shape, and temperature and have the same surface compliance but differ from each other either by weight or by texture.

Depending on the model adopted for test objects, a-weight may be inserted in the center of the object to ensure that the center of mass is located in the middle of each object. To minimize potential feedback regarding an object's contents (for example, if it is violently shaken or dropped), the contents should not shift or move. Clay and ping-pong balls, or other comparable materials, are useful to ensure that the weights are secured in the center of the test objects. To ensure that items in each of the weight groups (shown, for example, in Table 1 as groups 4-6) are indeed essentially identical weight matches, the objects are weighed on a balance scale as opposed to a spring-loaded scale.

According to the present example, textured coverings are applied to the vertical surface of the pipe and not to the lids. Essentially identical lids are glued to both ends of the pipe pieces. The textural materials are selected to mimic as closely as possible common surfaces. Examples of such textured surfaces include plastic, paper, and Styrofoam, such as the variety of surfaces found on disposable drinking cups. Specific materials that may be used to mimic such surfaces include 4 inch clear plastic packing tape, construction paper, and cork gasket lining. To ensure that there is no variation due to the thickness of any of the textured surface materials, all test objects are covered with the thickest of the materials (for example, cork, which is 1/16 inch thick). The objects may also be covered with each of the other materials if the thicknesses of the same are not negligible; in each case, the final covering on each individual test piece will vary based upon its grouping. Textured coverings are applied so as to minimize the profile of the seams to minimize additional or conflicting feedback.

Active Sensation Test Administration

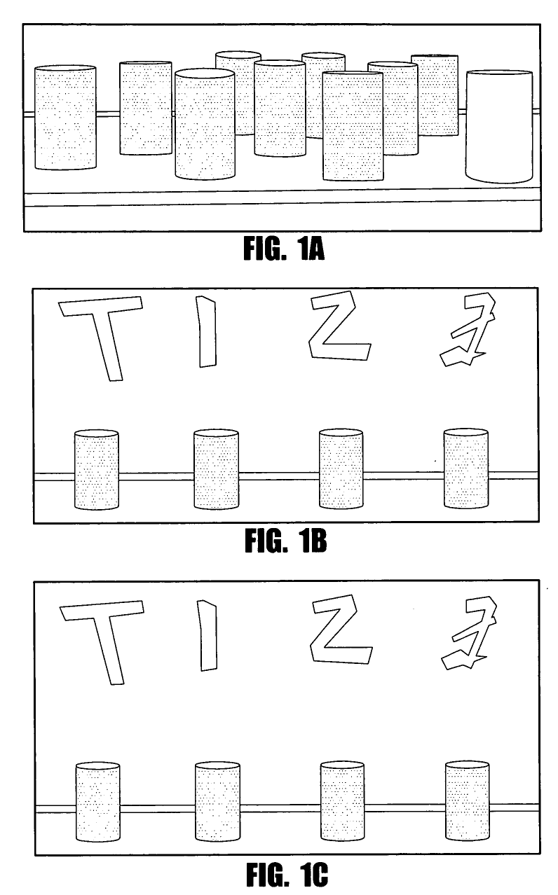

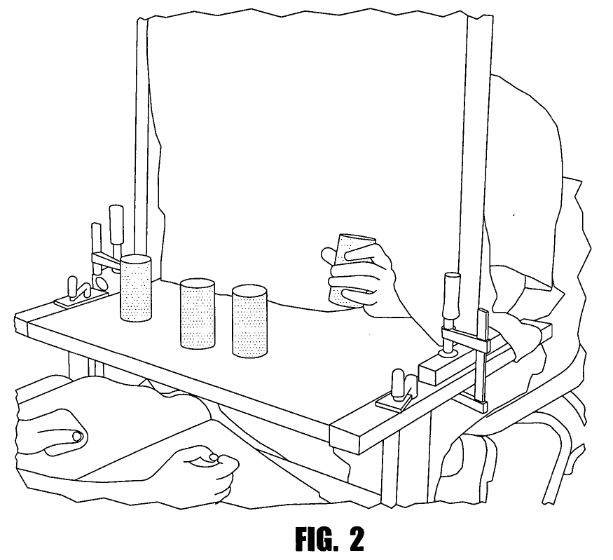

Test subjects are seated at a table having a height that permits them to rest and move the tested arm comfortably on the table, as depicted in FIG. 2. A barrier is erected that prevents the test subject from viewing either the test objects or the test examiner. For example, a curtain or the like may be mounted to and situated over the table, the curtain having the following labels printed on it reading from left to right: “T” for target, “1,” “2,” and “3” to indicate the order and the locations of the objects behind the curtain. (See FIG. 1, panels A, B, and C). According to this example the subjects sit on the labeled side of the curtain, and the examiner sits on the opposite side with the objects in front of the examiner and just behind the curtain. The curtain prevents the subjects from seeing the tested arm, the objects, and the examiner. The subjects place the test arm under the curtain. Subjects having or suspected of having nervous system impairment should use the more affected upper extremity to perform the test.

Subjects are instructed to find the match for the target object among the other three objects. It is important to note that they are not asked to match to a particular object property (e.g. “find the object that has the same texture), nor are they asked to identify the object or describe why they chose the match. Subjects are instructed that everything about the objects is the same except for the weight and texture, and that after first feeling the target object, they are permitted to feel the target and match objects as many times as they wish and in any order they chose. Subjects are informed that they will be measured by accuracy and by time—how long it took them to find a match and that the examiner cannot offer any feedback during the testing trials.

After the test procedure is explained, subjects are given two demonstration trials of the test with sample objects. The sample objects are not the same size as the test objects nor do they have the same distinctions as the test objects. The first test trial is to match the weight of objects with the same texture and the second test trial is to match the texture of objects with the same weight. For example the target weight may be 3 ounces and the matching weights may be, in order of presentation behind the curtain, 6 ounces, 1.5 ounces, and 3 ounces. The target texture may be white copy paper, and the matching textures may be, in order, 400 grade sandpaper, white copy paper, and glossy cardboard. After the demonstration trials, subjects are asked if there are any questions and clarifications to the instructions are made.

After the demonstration trials, subjects are given two actual trials. For each trial, the examiner places the objects in the appropriate locations behind the curtain. These locations correspond to the labels on the curtain but are tailored to the location of the subject's arm and amount of reach as observed during the two demonstration trials. When the trial objects are in place, the examiner asks the subject if he or she is ready to find the match. If the subject indicates yes, the examiner says: “Go.” The subjects explore the target object first using the more involved hand, as instructed, and then the potential matches. If elbow or shoulder movement prevents a subject from moving between objects, the examiner can slide the objects over to and away from the subject's hand per the subject's request. Once the subject finds the match, he or she indicates to the examiner either verbally by saying the number of the match, tapping the object they choose, or pointing at the number on the curtain.

Collection of Data: Scoring

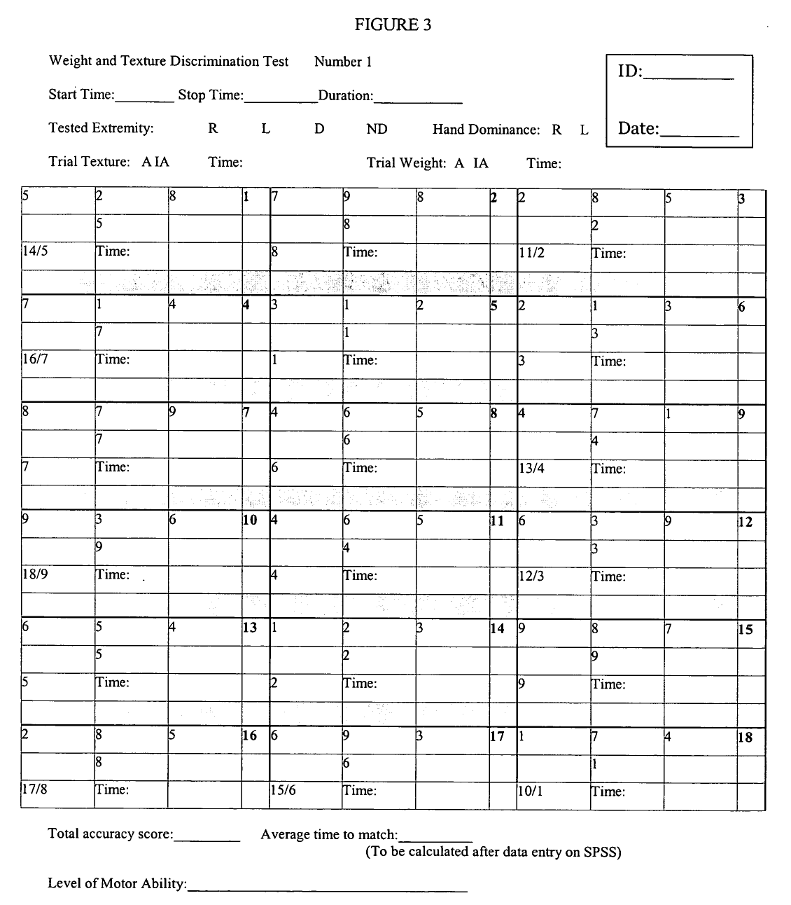

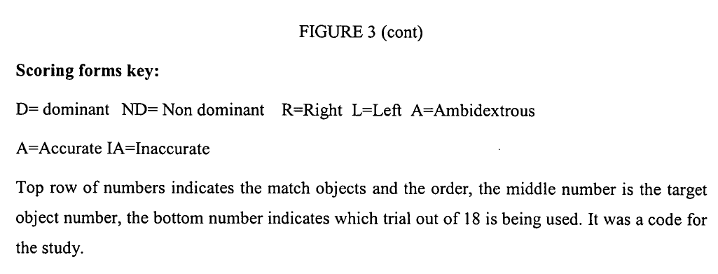

Using a score sheet such as that shown in FIG. 3, the examiner records the choice of the match and the amount of time the subject requires to make the match for each of the 18 trials on the Active Sensation Test. The stopwatch starts when the subject touches the target object and stops when the subject stops exploring and indicates the matching object. A separate clock measures Active Sensation Test duration. Test duration reflects the total time it takes to administer the 18 trials as it includes not only the subject's exploration time but also the time to set up and take down each trial and any rest breaks requested by a subject. The overall accuracy score for the Active Sensation Test is calculated after the test by adding the correct number of matches. Average time to match for each subject on the Active Sensation Test is calculated by adding all of the recorded times and dividing by the number of trials.

Index of Impairment

An average error score is calculated for each test subject in order to provide a discrete quantitative score. The average error score is calculated by adding together the error in the overall accuracy score for each test subject and dividing by the total number of trials. A distribution is then determined for all test subjects in order to identify cutoffs for degree of impairment.

Level of motor ability is determined by observing the variety of exploratory movements a subject employs during the trials. Low motor ability subjects use only grasp, lift, and release and are unable to use additional exploration movements. These subjects also need the objects placed near the hand and removed upon request. Medium ability subjects perform one exploratory pattern in addition to grasp, lift and release: either unsupported holding/hefting or lateral motion with at least the thumb and the index finger. Subjects who use both unsupported holding and lateral motion to explore the objects independently and simultaneously with grasp, lift, and release are classified as high motor ability.

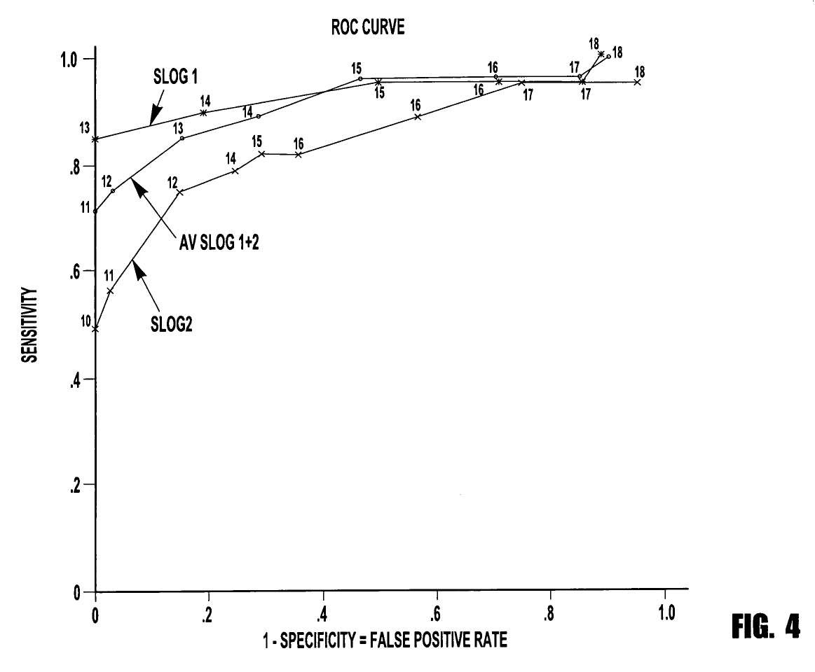

Referring to FIG. 4, Receiver Operating Characteristic (ROC) Curves using normalized sensitivity and specificity values for AST overall accuracy scores were created using data generated for a heterogenous sample of stroke survivors, compared to age, gender, hand dominance, and hand tested matched controls. Using the average of session scores, an AST criterion of impairment of less than 13 has a sensitivity value of 0.857 and specificity of 0.857 and the highest value for correct classification rate: 0.857. Subjects with a score of 12 or 14 fall into the zone of uncertainty where impairment may be mild. Scores above 14 suggest no impairment, whereas scores below 12 through 1 indicate moderate to severe impairment. Subjects scoring 11 and 10 can be considered moderately impaired, and subjects scoring less than or equal to 9 are more severely impaired. Subjects scoring 14 may be considered mildly impaired, while subjects scoring 15 are in the outer limits of the confidence interval, and are most likely non-impaired, and subjects scoring 16 or greater are definitively non-impaired.

The AST score not only offers a criterion score of impairment vs. non-impairment, but also provides a quantitative index score with a high resolution of gradation of impairment that can be used to track improvements as a result of intervention or recovery. The current index can be used for stroke survivors, and most likely for other individuals with central nervous system impairment due to injury such as traumatic brain injury.

Identification of Lesion Location

The AST overall accuracy score does discriminate among stroke survivors based upon lesion location—the variability in stroke survivor scores was statistically attributed to lesion location and not to date of injury, side of injury, level of motor ability, age, or gender. Low scores, or scores indicating impairment, are consistent with injuries to the somatosensory processing system whereas higher scores support the finding that the somatosensory system remains intact/is not injured. The larger the injury or the more targeted to the somatosensory system, the lower the AST overall accuracy score. This finding contributes to the construct validity of the AST and offers users pertinent information regarding the impairments associated with a particular lesion that will inform intervention strategies.

### EXAMPLES

### Example 1

### Evaluation of Stroke Survivors by the Active Sensation Test

The interpretation of the average error score on the Wrist Position Sense Test (WPST) employed by Carey et al provides an example for interpreting the overall accuracy score on the Active Sensation Test. The average error score is calculated by adding the total error for each subject and dividing by the total number of trials. In the WPST model, subjects were evaluated for performance on each of the 20 trail angles for wrist position, and were given an average error score calculated by summing the error values for each of the 20 angles (calculated per test angle by finding the difference between the test angle and the angle indicated by the test subject) and dividing by 20. The results provided an average error score for controls of 6.1° with standard deviation of 1.8° and a range of 3.1° to 10.9°. All of the scores for the controls were below 11°, so they defined a conservative 100^{th} percentile criterion for abnormality as 11° with a zone of uncertainty of 6.2° to 15.8.° Impaired scores were unmistakably identified above this range and unimpaired below it. While this interpretation does offer the examiner a cutoff for impairment, the WPST gives the examiner quantitative scores of proprioceptive performance with relatively fine resolution that have previously not been available. To limit the scores to “impaired” vs. “non-impaired” decreases the value the WPST offers the examiner about individual subject performance.

Applying the WPST approach for error analysis, test subjects were evaluated using the Active Sensation Test. Stroke survivors had a significantly lower overall accuracy score and a significantly longer average time to match than the control subjects (p<0.001). The distribution of the control subjects overall accuracy scores was positively skewed as all of the control subjects scored 13 or higher. The mean score was 14.86 with a standard deviation of 1.53 and 95% confidence interval 14.28-15.44. This is to be expected as the control group was hypothesized to be unimpaired and, therefore should have less variability in their scores. Stroke survivors had far more variability and followed a more normal score distribution with accuracy scores varying from 1 to 17. The mean score was 8.46 with a standard deviation of 3.51 and 95% confidence interval of 7.14-9.88. Twenty-one or 75% of the stroke survivors scored between 6 and 11, with only four stroke survivors scoring 13 or higher and three scoring 4 or lower.

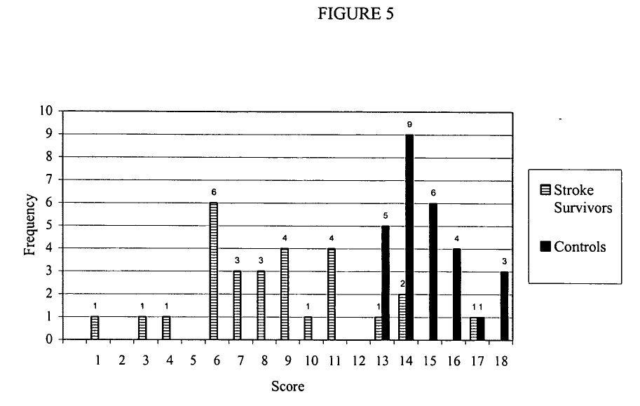

Referring to FIG. 5, results of testing are shown in a ROC curve. The results indicate the relationship between true positive rate or sensitivity and false positive rate or 1-specificity. The ROC table (Table 2) provides a means of assessing the ability of a test to discriminate between impaired and non-impaired subjects at various scores. Sensitivity is the ability of the test score to correctly identify subjects with an impairment and specificity is the ability of the test score to correctly identify non-impaired subjects as non-impaired. Positive predictive power reflects the likelihood that the subject is truly impaired if the test score indicates impairment. The opposite is true for negative predictive power. Using 13 as the cutoff has the highest correct classification rate for session 1 scores as well as for the average of the two session scores. Sensitivity of 0.857 and specificity of 0.857-1.00, a positive predictive power of 0.857-1.0, false positive rate of 0-0.143 and a false negative rate of 0.143. The values of 12 and 14 are not largely different from 13, meaning that 13 can be considered the criterion of impairment with a zone of uncertainty from 12 to 14. Subjects scoring 11 and 10 can be considered moderately impaired, and subjects scoring less than or equal to 9 are more severely impaired. Subjects scoring 14 may be considered mildly impaired, with subjects scoring 15 are in the outer limits of the confidence interval and most likely non-impaired and subjects scoring 16 or greater are non-impaired.

TABLE 2
ROC TABLE

| AST Score session 1 | = 12 | = 13 | = 14 | = 15 | = 16 | = 17 | = 18 |
|---|---|---|---|---|---|---|---|
| Correct | .923 | .923 | .857 | .732 | .625 | .554 | .554 |
| Classification Rate | | | | | | | |
| Sensitivity | .857 | 857 | .893 | .964 | .964 | .964 | 1.0 |
| Specificity | 1.0 | 1.0 | .821 | .500 | .286 | .143 | .107 |
| False + Rate = 1 − Specificty | 0 | 0 | .179 | .500 | .714 | .857 | .893 |
| False − Rate | .143 | .143 | .107 | .0356 | .036 | .036 | 0 |
| +Predictive Power | 1.0 | 1.0 | .833 | .659 | .575 | .529 | .528 |
| −Predictive Power | .875 | .875 | .885 | .933 | .889 | .800 | 1.0 |

| AST Score session 2 | = 10 | = 11 | = 12 | = 13 | = 14 | = 15 | = 16 | = 17 | = 18 |
|---|---|---|---|---|---|---|---|---|---|
| Correct | .750 | .768 | .804 | .768 | .768 | .732 | .661 | .607 | .500 |
| Classification Rate | | | | | | | | | |
| Sensitivity | .500 | .571 | .750 | .786 | .821 | .821 | .893 | .964 | .964 |
| Specificity | 1.0 | .964 | .857 | .750 | .714 | .643 | .429 | .250 | .036 |
| False + Rate = 1 − Specificty | 0 | .036 | .143 | .250 | .286 | .357 | .571 | .750 | .964 |
| False − Rate | .50 | .429 | .250 | .214 | .179 | .179 | .107 | .036 | .036 |
| +Predictive Power | 1.0 | .941 | .840 | .759 | .742 | .697 | .610 | .563 | .500 |
| −Predictive Power | .667 | .692 | .774 | .778 | .80 | .783 | .80 | .875 | .500 |

| AST Score average 1, 2 | = 11 | = 12 | = 13 | = 14 | = 15 | = 16 | = 17 | = 18 |
|---|---|---|---|---|---|---|---|---|
| Correct | .857 | .857 | .857 | .804 | .750 | .625 | .554 | .554 |
| Classification Rate | | | | | | | | |
| Sensitivity | .714 | .750 | .857 | .893 | .964 | .964 | .964 | 1.0 |
| Specificity | 1.0 | .964 | .857 | .714 | .536 | .286 | .143 | .107 |
| False + Rate = 1 − Specificty | 0 | .036 | .143 | .286 | .464 | .714 | .857 | .893 |
| False − Rate | .286 | .250 | .143 | .107 | .036 | .036 | .036 | 0 |
| +Predictive Power | 1.0 | .955 | .857 | .758 | .675 | .575 | .529 | .528 |
| −Predictive Power | .778 | .794 | .857 | .870 | .938 | .889 | .800 | 1.0 |

The classification table (Table 3) place 4 stroke survivors in the control group classification further support this interpretation of the accuracy score. If the control group can be considered “non-impaired” then those four stroke survivors are also considered non-impaired, while the remaining stroke survivors are considered impaired. One of the stroke survivors scored above 14, the upper limit of the zone of uncertainty, with an accuracy score of 17, while none of the control subjects scored below the lower limit of 12.

TABLE 3
Classification Table for predicted group membership Active Sensation Test
accuracy and average match time scores

| | | Predicted | | | Average Match | | Predicted | | |
|---|---|---|---|---|---|---|---|---|---|
| Overall Accuracy | | GROUP | | | Time | | GROUP | | |
| Observed | | SS | Controls | % Correct | Observed | | SS | Controls | % Correct |
| GROUP | SS | 24 | 4 | 85.7 | GROUP | SS | 19 | 9 | 67.9 |
| | Controls | 0 | 28 | 100.0 | | Controls | 6 | 22 | 78.6 |

| | | | |
|---|---|---|---|
| Overall % | 92.9 | Overall % | 73.2 |

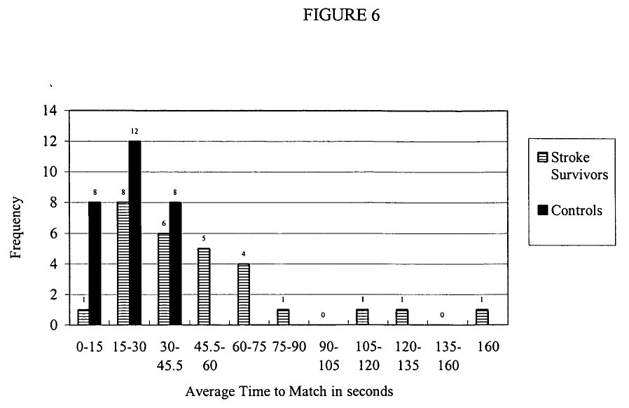

The second score, average time to match, although significantly different for the two groups, appears to be less discriminating on its own as just over half of the stroke survivors had similar time scores as the non-impaired controls. If the same decision process of choosing the time above which no controls subjects scored were used to distinguish impaired from non-impaired subjects for average time to match, then 15 of the 28 stroke survivors could be considered non-impaired. Referring to FIG. 6, average times to match for the control group were normally distributed and were all below 45.50 seconds. The mean was 23.68 seconds and the standard deviation was 9.99 seconds with a 95% confidence interval of 19.98-27.38. The distribution of the stroke survivors' average times to match again demonstrated more variability with a range of 14.39 to 157.97 seconds. They were positively skewed with 54% of the subject set scoring below 45.50 seconds. The mean average time to match for the stroke survivors was 52.50 with a standard deviation of 35.37 seconds and 95% confidence interval of 39.40-65.60. Only 7 stroke survivors had an average time to match below the upper limit of the control subject 95% confidence interval, 27.38 seconds. Three of the controls had an average time to match within the stroke survivor 95% confidence interval.

The classification table for average time to match reflects this lack of discrimination as it placed 6 controls and 19 stroke survivors in the impaired group with a cut off time of approximately 32.50 seconds. Average time to match may be more variable as a performance indicator because every subject, stroke survivor and control, used different strategies to search the objects. Average time to match did not differ significantly by level of ability for the stroke survivors on one-way ANOVA (F=0.188, p=0.830), and the frequencies of the average time to match were evenly distributed across the spectrum for all three ability levels.

Of the 19 stroke survivors placed in the impaired group by the classification table, 8 were in the high ability group, 4 from the medium, and 6 from the low ability. A high ability stroke survivor scored the longest average time to match, 157.97 seconds. The choices in search strategies, and thus the differences in average time to match, could have been influenced not only by level of motor ability and experience of fatigue but also by such personality characteristics as cognitive level, decision making ability, competitiveness, perfectionism, and impatience. The differences in average time to match seem to be influenced by variables other than sensibility.

Regardless of how items on the test were divided—by object property, by property quality, or by individual trial—all of the comparisons between stroke survivors and controls revealed significant differences for both overall accuracy and average time to match except for two individual test trials out of the 18 total. (Tables 4 and 5). Paired sample t-tests were used to compare texture perception to weight perception by looking for significant differences between scores on the 9 weight trials vs. the 9 texture trials. The within test comparison's found a significant difference in overall accuracy scores, but not for average time to match, with all of the subjects scoring higher on the texture trials as opposed to the weight trials (t=3.349, p=0.001). The mean score for the texture trials was 6.36 with a standard deviation of 2.497 and the mean score for the weight trials was 5.30 with a standard deviation of 2.311. When the same within test comparisons were run for the stroke survivors and the controls, the same relationships, although less powerful (p<0.05), were found for both scores for the control subjects and only the accuracy score for the stroke survivors. Stroke survivors scored 4.75 correct for texture and 3.71 for weight and the controls scored 7.96 for texture and 6.89 for weight. These results suggest that the texture trials were easier to match than the weight trials. Indeed some of the subjects, when actively exploring during a weight trial, stated that “these trials” are more difficult or had “more subtle differences.” Average time to match between texture and weight was only significantly different for the control group, while overall accuracy was discriminative for the entire subject set as well as both groups.

Because of the chance that stroke survivors would have an overall accuracy score equivalent to their matched controls, average time to match was included as a separate measure of performance. The thought was that if the accuracy scores were the same, the average time to match would provide the distinction. In other words, the average time to match might distinguish between individuals with the same overall accuracy score. This premise was investigated by correlating the average time to match with overall accuracy score. The Pearson r value was negative and moderate in strength, −0.414 with p equaling 0.002 for the entire 56 subject set. When the two scores were correlated for the stroke survivors and the control group, the relationship between the scores was no longer present for either group. Additionally, for the 32 subjects out of the 56 who scored 13 or higher for overall accuracy, Pearson r did not reveal a significant correlation between accuracy and average match time. The same result was found for the 24 subjects who scored below 13. The four stroke survivors who scored 13 or higher did not have the longest average times to match when compared to the control subjects who had the same scores. When both scores were combined in another binary logistic regression classification table, the same distribution was found as that for the accuracy score-all of the controls and four of the stroke survivors were considered non-impaired. These results seem to suggest that while there were significant differences between stroke survivors and controls for average time to match, the overall accuracy score is a more sensitive measure of impairment in general.

In order to be able to include the spectrum of subjects with nervous system impairment, such as stroke survivors, including those with either acute or chronic injury, all participants in the study were tested and retested on the Active Sensation Test during the same day. This was done to limit the influence of recovery on the second test scores. Most other studies that evaluated the test-retest reliability of sensory tests did not test subjects during the same day; rather, they had 24-hours up to a few weeks break time between testing sessions. Another limitation to the same day testing is that most tests are not administered on the same day in the clinical setting. The second rationale behind same day testing was to limit the potential influence of practice of test-like conditions or therapy interventions between testing sessions. Break time duration varied between 45 minutes up to 4 hours—being driven by the schedule of the stroke survivor. Break times for the control subjects were equal to the break duration of the stroke survivor to whom they were matched. Subjects spent the break time having lunch, running errands, reading books, napping, participating in a sporting event (controls) or participating in a non-related therapy activity (e.g. gait training).

In summary, the results of the analyses comparing the stroke survivors to the controls support the validity of the Active Sensation Test to distinguish individuals with and without a feeling impairment. In addition, not only does the overall accuracy score offer an objective criterion for judging impaired vs. non-impaired, it also provides a quantitative measurement of object property perception. The stability of the results between the two groups when the Active Sensation Test items were separately compared suggests that the test objects and the properties selected were effective.

TABLE 4
Weight and Texture Discrimination Test Overall Accuracy Scores by object
property, property gradient, and per trial for both groups

| Property | | | Gradient | | | Trial | | | Mean | |
|---|---|---|---|---|---|---|---|---|---|---|
| 9 trials | t | p | 3 trials | t | p | 1 trial | t | p | SS | Mean C |
| Texture | −6.277 | .000 | Paper | −5.658 | .000 | 3 | −2.741 | .009 | .54 | .86 |
| Mean | 4.75 | | | Mean | 1.32 | 12 | −4.379 | .000 | .36 | .86 |
| SS | | | | SS | | | | | | |
| Mean C | 7.96 | | | Mean C | 2.71 | 18 | −6.000 | .000 | .43 | 1.00 |
| | | | Cork | −3.550 | .001 | 1 | −1.137 | .261 | .61 | .75 |
| | | | | Mean | 1.54 | 9 | −2.951 | .005 | .46 | .82 |
| | | | | SS | | | | | | |
| | | | | Mean C | 2.43 | 17 | −3.351 | .002 | .46 | .86 |
| | | | Plastic | −3.998 | .000 | 4 | −3.250 | .003 | .64 | .94 |
| | | | | Mean | 1.89 | 10 | −2.729 | .009 | .64 | .93 |
| | | | | SS | | | | | | |
| | | | | Mean C | 2.82 | 16 | −3.025 | .004 | .61 | .93 |
| Weight | −7.080 | .000 | 6 ounces | −4.430 | .000 | 5 | −1.455 | .152 | .61 | .79 |
| Mean | 3.71 | | | Mean | 1.57 | 7 | −2.278 | .027 | .64 | .89 |
| SS | | | | SS | | | | | | |
| Mean C | 6.89 | | | Mean C | 2.54 | 11 | −4.770 | .000 | .32 | .86 |
| | | | 7 ounces | −4.017 | .000 | 2 | −2.510 | .015 | .39 | .71 |
| | | | | Mean | 1.14 | 13 | −2.496 | .016 | .32 | .64 |
| | | | | SS | | | | | | |
| | | | | Mean C | 2.14 | 14 | −2.540 | .014 | .43 | .75 |
| | | | 8 ounces | −5.232 | .000 | 6 | −2.496 | .016 | .36 | .68 |
| | | | | Mean | 1.00 | 8 | −4.257 | .000 | .29 | .79 |
| | | | | SS | | | | | | |
| | | | | Mean C | 2.21 | 15 | −3.161 | .003 | .36 | .75 |

KEY:
SS—Stroke Survivors;
C—Controls

TABLE 5
Weight and Texture Discrimination Test Average Time to Match by object
property, property gradient, and per trial for both groups.

| Property | | | Gradient | | | Trial | | | | |
|---|---|---|---|---|---|---|---|---|---|---|
| 9 trials | t | p | 3 trials | t | P | 1 trial | t | p | Mean SS | Mean C |
| Texture | 4.339 | .000 | Paper | 4.277 | .000 | 3 | 4.043 | .000 | 49.35 | 24.55 |
| Mean | 50.23 | | | Mean | 50.16 | 12 | 3.521 | .001 | 54.55 | 23.13 |
| SS | | | | SS | | | | | | |
| Mean C | 21.63 | | | Mean C | 22.22 | 18 | 4.558 | .000 | 46.59 | 18.97 |
| | | | Cork | 3.975 | .000 | 1 | 4.714 | .000 | 53.59 | 24.46 |
| | | | | Mean | 55.81 | 9 | 3.554 | .001 | 61.51 | 22.72 |
| | | | | SS | | | | | | |
| | | | | Mean C | 23.86 | 17 | 2.528 | .014 | 52.32 | 24.41 |
| | | | Plastic | 3.908 | .000 | 4 | 3.339 | .002 | 42.63 | 21.62 |
| | | | | Mean | 44.74 | 10 | 5.480 | .000 | 42.71 | 17.27 |
| | | | | SS | | | | | | |
| | | | | Mean C | 18.82 | 16 | 2.692 | .009 | 48.86 | 17.57 |
| Weight | 3.586 | .001 | 6 ounces | 3.804 | .001 | 5 | 3.225 | .002 | 54.47 | 23.97 |
| Mean | 54.76 | | | Mean | 52.32 | 7 | 3.558 | .001 | 53.02 | 22.02 |
| SS | | | | SS | | | | | | |
| Mean C | 25.73 | | | Mean C | 22.14 | 11 | 3.674 | .001 | 49.48 | 20.42 |
| | | | 7 ounces | 3.131 | .004 | 2 | 3.354 | .001 | 64.74 | 29.36 |
| | | | | Mean | 57.67 | 13 | 2.736 | .008 | 52.64 | 29.69 |
| | | | | SS | | | | | | |
| | | | | Mean C | 28.87 | 14 | 2.466 | .017 | 55.64 | 2257 |
| | | | 8 ounces | 3.513 | .001 | 6 | 2.457 | .017 | 51.09 | 25.32 |
| | | | | Mean | 54.30 | 8 | 3.268 | .002 | 53.59 | 27.16 |
| | | | | SS | | | | | | |
| | | | | Mean C | 26.18 | 15 | 3.583 | .001 | 58.20 | 26.05 |

KEY:
SS—Stroke Survivors;
C—Controls

### Example 2

### Test-Retest Reliability

Reliability for sensory tests is frequently measured by a Pearson correlation with a zero-to-one scale where 1 means perfect reliability. The Pearson r indicates how predictive the two scores are for each other. While perfect reliability is nearly impossible to achieve in any testing situation, some authors suggest that reliabilities greater than 0.64 are acceptable where others recommend 0.80 or more. ^{25,26} The magnitude of the Pearson r can be influenced by variability and measurement error. In order to control for this effect, the Intraclass Correlation Coefficient with the same zero-to-one criteria, takes these challenges into consideration and calculates a correlation coefficient with systematic error increases or decrease in scores accounted for. The single measure ICC reflects how stable a single score is between test 1 and test 2 and the average measure ICC evaluates the average of the two session test scores essentially canceling out the influence of measurement error, chance, and luck may have on test scores. Although the value of the single ICC will be less than the Pearson r, the magnitude of the difference between the values will reveal if there is a systematic difference in the means. Based upon the calculation method of the average ICC, the value may be greater than that of Pearson r. Some authors report a kappa coefficient as this statistic is most beneficial when comparing item to item. Kappa is judged with the same zero-to-one criteria. Usually in sensory testing, test-rest reliability depends upon the skill of the examiner to administer the test consistently—stimulus application control. The results reported generally discuss inter-rater and intra-rater reliability, with intra-rater generally higher than inter-rater reliability.

The Active Sensation Test was designed to minimize the threats to inter-rater and intra-rater reliability by making every effort to eliminate the need for a skilled examiner to apply a controlled stimulus: the examiner had to place the objects in the correct order and operate a stopwatch. Hence, the threat to reliability for the Active Sensation Test are more likely due to the design of the test objects themselves, meaning that the test may be learnable. Although the same 9 objects were used in both tests, to limit the potential for learning, the 18 trials were randomly assigned for each test session and the order of the objects within each trial were also randomly assigned per session. This means that when object number 1 was to be matched for its weight, it was trial 5 in the first test session and trial 11 in the second test session. In addition, the order of the three potential match objects was different between the two test sessions.

A majority of the subjects found the match to sample activity of the Active Sensation Test to be both a challenging and novel task. Comments like “this is really interesting,” “I've never done anything like this before,” “this is difficult to do,” and “I am sure I haven't made one correct match” were common. At the same time, several subjects, also found the test to be tedious and boring—especially for those stroke survivors who took 30 or more minutes to complete the test and for the control subjects during the second test. The novel aspect of the test had “worn off” at that point and many of the subjects wanted to get the second test “over-with” so they could go home or do something else. The Active Sensation Test was the only test completed twice in this study—so the influence of these variables cannot be investigated on the stability of WPST or 2-point discrimination measures.

Test-retest reliability of the Active Sensation Test scores between the first and second administrations was supported by the significant relationships found between the two scores for the entire subject set as well as for the stroke survivor and control subgroups (p<0.01 and p<0.001). The nature of the relationships was positive and the magnitude varied from substantial to strong. Additionally, there was an interesting and persistent distinction between overall accuracy and average time to match. All of the relationships for average time to match were greater than r=0.850 while the relationships between the two overall accuracy scores were more variable, depending upon the independent variable used to group the subjects. Differences between the overall accuracy scores varied between significant and insignificant depending upon the comparisons (p<0.05). Change scores calculated for overall accuracy followed the same distributions as the first Active Sensation Test accuracy scores did. Conversely, all of the average match times dropped significantly during the second test for each comparison. Change scores were not calculated for average time to match secondary to the consistency in the differences found on the paired t-tests.

The strength of the relationship for the entire 56 subject set between the first and second overall accuracy score can be considered strong with a Pearson r of 0.777 and p value of 0.000. The single ICC value was 0.767, indicating each single measure was reliable between first and second test sessions. The average ICC was 0.868 suggesting that averaging scores was an even more reliable predictor of performance or that repeated testing enhances the discriminative value of the Active Sensation Test as opposed to diminishing the information the test score provides. Although the difference between the two test scores was found to be significant at the 0.05 level, the mean difference was only 0.82 points. The change scores varied from a drop in 4 points to a gain of 8 points with an average of a 0.8214 point increase. Standard error of measurement for the entire subject set (n=56) was 1.96 for the first test session and 1.70 for the second test. These values support that changes in score greater than 3 are most likely not due to measurement error. Taken together with the ROC interpretation, a change in AST score greater than 3 can most likely be attributed to a genuine change in performance that may be due to recovery or therapeutic intervention as opposed to measurement error.

Both the WPST and 2-point discrimination measures have published reliability data. Carey et al report the reliability of the WPST test to be between 0.88 and 0.92—well above the recommended values for reliability coefficients. Carey et al tested their subjects three times within 24-72 hour intervals. No other authors have corroborated their results. Depending upon the study and the method employed to test 2-point discrimination, the reliability reported varies from kappa=0.57 to kappa=0.18. Dellon, the designer of the DiskCriminator™, reports inter-rater reliability for 2-point discrimination with the DiskCriminator™ as r=0.92 with 87% of the measurements differing by 1 mm or less. No other authors have corroborated these results. Other authors who have designed tests of weight or texture discrimination inconsistently report reliability measures. Caselli, in his reports on tactile agnosia, does not report reliability measures for his tests of weight and texture discrimination. Carey reports that the Tactile Discrimination Test had high test-retest reliability although a specific r value was not reported. Gaubert et al recently evaluated the inter-rater reliability between three examiners on the stereognosis component of the Nottingham Sensory Assessment. The assessment uses 9 objects and the examiner scores the subject on a three point scale: normal, impaired, absent. They tested both the affected and non affected sides within a 24-hour period and compared the scores per item using a kappa coefficient. They found values indicating substantial agreement between examiners on most of the 9 items; however the scores for the affected side were in the fair to moderate range as compared to the non-affected side. They attributed the discrepancy to the scoring method and the testing method—if the subject was unable to handle the object, the examiner was permitted to move the object in the subject's hand. While these other sensory tests provide some guidance for interpreting the reliability results found for the Active Sensation Test, the testing methods and scoring do not match those used for the Active Sensation Test.

### Example 3

### Correlation of Active Sensation Test Scores with Lesion Location

The variability among stroke survivors' scores was not attributed to time since injury or personal report of loss of sensation as significant differences were not found between their scores based upon those criteria. The variability among the stroke survivors' scores was expected and hypothesized to be related to the location of the stroke lesion. Lesion locations were classified by suspected impact on the somatosensory system. These locations were built around the neuroanatomical data referenced for haptic touch transmission along with symptoms reported by prior studies for various stroke lesions. Semmes et al adopted a similar classification strategy in their 1960 study of palm 2-point discrimination thresholds for men who had sustained penetrating brain wounds during World War II and the Korean War. Their divisions were related to impact on the cortex, specifically the contralateral sensorimotor cortex, the ipsilateral sensorimotor cortex, and subjects with cortical injuries that spared the sensorimotor cortex. For this study, subjects were divided into five categories: sensorimotor cortex injury (sm cortex), cortex injury sparing the sensorimotor area (cortex non sm), internal capsule/thalamus injury (int c/thalamus), lacunar/basal ganglia injury (lacunar/BG), and injury to the pons region. Because all of the locations were above the medulla and thus, the internal arcurate fiber tract that brings the ascending somatosensory information carried by the dorsal columns in the spinal cord to the contralateral side of the brain, all lesions were considered contralateral to the affected upper extremity.

TABLE 6
Active Sensation Test score comparisons among stroke survivor subjects.

| | | | | | Post | Hocp |
|---|---|---|---|---|---|---|
| Comparisons | Accuracy Score Match Time | | | | Scheffe | value |
| | F Value | p value | F Value | p value | | |
| Lesion location | [symbol] | [symbol] | 1.041 | .408 | *SM vs | [symbol] |
| | | | | | non SM | |
| Level of Ability | [symbol] | [symbol] | .188 | .830 | *High | [symbol] |
| | | | | | vs Low | |
| Time Since Injury | .665 | .523 | 1.151 | .332 | | |
| Lesion × Ability | 2.676 | .078 | n/a | n/a | | |
| Gender × Ability | n/a | n/a | 2.293 | .125 | | |
| | t value | p value | t value | p value | | |
| Hand Tested | .371 | .714 | .402 | .691 | | |
| Hand Dominance | −.661 | .540 | .788 | .438 | | |
| Perception of | 1.493 | .147 | 1.541 | .135 | | |
| Loss | | | | | | |
| Gender | 1.985 | .058 | [symbol] | [symbol] | | |

KEY:
Significant differences in bold/italics.
*indicate post hoc differences for that comparison.
SM = Sensorimotor cortex;
non SM = injuries to the cortex that spare the sensorimotor area.
n/a—analysis not applicable.

A one-way ANOVA revealed a significant difference between stroke survivors' overall accuracy score by lesion location, but not for average time to match (p<0.05). The post hoc analysis found a significant difference between subjects with sensorimotor cortex lesions and cortical injuries that did not affect the sensorimotor cortex (p<0.05). The average accuracy score for subjects with sensorimotor cortex injuries was 5.90 with a standard deviation of 2.079 as opposed to 11 and a standard deviation of 1.915 for subjects with cortical injuries that spared the sensorimotor cortex. These two subgroups had the most subjects: 10 in the sensorimotor cortex and 7 in the non-sensorimotor cortex injuries. Although this result needs to be interpreted with caution as two of the five classes had only three subjects, that this small sample of 28 stroke survivors demonstrated a significant difference at the p<0.05 level suggests that the Active Sensation Test does have the potential to discriminate among lesion locations of stroke survivors.

As the primary somatosensory and primary motor cortices for the contralateral hand are located in the sensorimotor cortex on the convexity of the frontal and parietal lobes, it can be expected that lesions in the contralateral sensorimotor cortex will have the greatest impact upon object property perception/haptic perception To briefly review, the S-I cortex interprets the single somatosensory dimensions of proprioception and cutaneous touch in order to discriminate texture and weight along with other object properties like size and shape. The primary motor cortex interprets the motor program, in the form of an exploratory procedure, into a sequence of activations and delivers it to the muscles via the corticospinal tract. As a group, the 10 stroke survivors in the SM cortex group had the lowest average score on the Active Sensation Test of 5.90 with a range of 1 to 8 correct. Although not significantly different between lesion location groups, the average time to match for the sm cortex group was 48.35 seconds with a range of 18.71 to 133.60 seconds. Average time to match does not seem to provide additional information to distinguish the sm cortex group members from each other as the two scores do not correlate significantly. Level of motor ability for this group was distributed across all three categories and did not correlate significantly with either overall accuracy score or average time to match. (See Tables 4.5)

The cortical lesions in this study that spared the sensorimotor cortex were focused either in and around the insula or at the junction of the parietal-occipital lobes. While these areas are reported to receive input from and deliver input to the sensorimotor cortex region, it was anticipated that they would not have a significant impact on object property perception as tested by the Active Sensation Test. Indeed, the mean score for the 7 subjects in cortex non sm group was among the highest at 11.00 with a range of 9 to 14 correct. All of these subjects were in the high motor ability category reflecting sparing of the frontal lobe motor regions by the injuries. The average time to match was 56.51 seconds with a range of 15.86 seconds to the highest value of 157.97 seconds. Two of the individuals considered non-impaired by overall accuracy score were from this group. Their average match times were 15.86 with 13 for accuracy and 41.12 seconds with 14 for accuracy.

Because the secondary somatosensory area is connected to the temporal lobe via the insular cortex and it has been implicated in object identification based upon prior experience, injuries to these areas would be expected to and have been reported to affect a subject's ability to identify the object or the combination of object properties that define it. During the Active Sensation Test, subjects were asked to complete a task: find a match through active exploration. They were not asked to identify the object property nor explain why they chose their match. This distinction from SM cortex injuries is reflected in the significantly higher overall accuracy scores. Lesions in the posterior parietal cortex are reported to affect motor planning as well as integration of visual and spatial information for body awareness. The influence of this injury was not possible to measure independently as only two subjects presented with lesions in the parietal-occipital region. One subject presented with mild left neglect that did not influence test participation. Neither subject demonstrated apparent motor planning problems.

The data for lesions in the thalamus/internal capsule region, basal ganglia, and pons can not be considered definitive results due to the small numbers of subjects in each group: 5, 3, and 3 respectively. Regardless, the scores seem to be representative of what could be expected for these types of injuries. Lesions in the thalamus and internal capsule are expected to affect object property perception as the ascending fibers of the medial leminiscal pathway synapse there or are the third order neuron that travels to S-I. Injuries in this region have the potential to influence the perception of discrete modalities of touch and proprioception as they interrupt the ascending connections to the somatosensory cortex. The average score for the 5 subjects in this category was 8.60 with a range of 6 to 11 correct matches. Average time to match was 76.77 seconds with a range of 51.67 to 119.90 seconds. Motor ability will also be affected by injuries in this area as the descending corticospinal tract fibers synapse or pass through these areas. None of the 5 subjects were classified in the high motor ability group.

Basal ganglia and lacunar infarcts are deep, subcortical injuries that are small and most likely will not have as strong of an effect on sensory perception as they might on motor ability.²⁰ The mean score for the 3 subjects in this group was 11.00—the same score as for the 7 individuals with cortical lesions that spared the sensoriomotor areas—with a range of 7 to 17. Additionally, these three individuals were all in the high motor ability group. The average time to match was 35.37 seconds with a range of 14.39 to 50.00 seconds. One of the four subjects considered non-impaired by accuracy score came from this group—the accuracy score was 17 with an average time of 50.0 seconds. Lesions in the pons have the potential to affect ascending and descending fibers—if the lesion is in the region of the tracts—producing variable effects. The scores for the three subjects with these injuries demonstrate this variability. Their average score was 8.33 with scores of 3, 8 and 14. The motor ability of the three subjects with lesions in the pons was distributed across all three levels—the low subject scored 8 with an average match time of 22.61 seconds. The fourth subject considered non-impaired by the overall accuracy score was in this group and was considered high ability, with an accuracy score of 14 and a match time of 21.04 seconds.

The level of motor ability or the variety of exploration possible for each stroke survivor could be expected to positively influence both average time to match as well as overall accuracy score especially since the four individuals considered non-impaired were all in the high motor ability group. Level of motor ability, like lesion location, did demonstrate a significant difference between stroke survivors for overall accuracy (F=5.428, p=0.011), but not for average time to match (F=0.188, p=0.830). The post hoc analysis identified a significant difference between high and low ability subjects (p<0.05). While the high ability subjects did have a higher mean score (10.46) than the low ability subjects (6.25) by 4.21 points, the two groups had similar ranges of scores. The minimum score for the low ability group was 1, with a high score of 11. The high ability subjects scored between 6 and 17. Univariate analysis did not reveal a significant interaction effect for motor ability and lesion location (F=2.676, p=0.078), nor a significant main effect for either criteria. The power for both lesion location and level of ability on the one-way ANOVA, while below the 0.05 level, was most likely not strong enough due to insufficient subject numbers; however, that an effect was detected on one-way ANOVA does make this an area worth pursuing in future evaluations of the Active Sensation Test.

Data about potential effects of gender, age, hand dominance, or hand tested on object property perception was not found in the literature. To ensure that the variability in scores was not due to these variables, even though the selection process controlled for them, the entire subject set as well as each group were compared based upon these independent variables. Across the entire 56 subject set, no significant differences were found in both scores due to gender, age group, hand dominance, hand tested or APHQ category. The control subject group was more homogeneous which was confirmed by insignificant differences in scores within the group due to age group, gender, hand dominance, hand tested or APHQ classification. The variability in the two scores within the stroke survivor group was not attributed to age group, hand dominance, hand tested or APHQ category. Only gender revealed a significant difference for the average time to match for the stroke survivors (p<0.05).

As there was a significant relationship identified between gender and ability, this interaction may have been the source for the difference in average time to match for men and women in the stroke survivor group. The follow-up univariate analysis of the interaction of ability and gender did not demonstrate a statistically significant interaction effect on average time to match (F=2.293; p=0.125), nor did it find a main effect for either gender or ability in the stroke survivor group. This result can be expected as the level of significance for the difference between men and women (t=−2.268; p=0.041) as well as for the strength and level of significance for the relationship between gender and ability (V=0.465; p=0.049) were not as high as for the other differences found in the study where p was less than 0.001 in most cases. While women were shifted towards lower function as compared to the men, this may be merely an artifact of this study sample. The National Stroke Association reports that while women account for only 43% of stroke incidence per year, they account for 61% of stroke deaths. Even though more women die from stroke per year, more women than men are living as stroke survivors. The NSA suggests that the explanation for these findings may be because stroke risk increases with age and that women generally live longer than men. Some European authors have reported that men have a better functional outcome than women and that more men than women are discharged to home. This last observation may be due more to the availability of caregivers for women as opposed to functional outcome. Future studies of the Active Sensation Test should investigate this potential interaction further in addition to investigating a relationship between gender and lesion location. Gender and lesion location interactions were not pursued, as there was insufficient representation within the groups.

The results of the within group comparisons between the stroke survivors seem to support the premise that the overall accuracy score is the more sensitive indicator of impairment as compared to average time to match. The significant effects of lesion location and motor ability on the overall accuracy score, and not average time to match, points to a greater discriminate potential of the overall accuracy score for stroke survivors. This correlation lends more credibility to the Active Sensation Test—that it indeed measures what it intends to measure. This thought is further supported by the lack of relationship between the two Active Sensation Test scores within the three subgroups of motor ability and within the three lesion location classes with 5, 7, and 10 subjects.

The distribution of scores across the low and high level of motor ability suggests that performance on the Active Sensation Test depends upon more that just the ability to perform exploratory movements. These results support the premise that the Active Sensation Test is indeed a distinct measure of hand performance, a different measure of hand function. The analysis across level of ability suggests that while individuals who are capable of performing more than grasp, lift and release may score higher on average than those who can only use grasp, lift, and release, the ability to perform grasp, lift and release is sufficient for taking the test. Additionally, it also suggests that level of motor ability is not the primary factor influencing test performance. The subjects with a high degree of motor ability may have more options for exploration, but merely having the ability to perform various exploratory procedures is not sufficient. The sensori-perception system needs to be able to integrate the multiple sensory messages gathered by the hand during the exploration into some form in order for the correct match to be made.

### Example 4

### Examination of Causes of Variation in and Reliability of Active Sensation Test Scores

To further investigate the relationships and differences between the scores to perhaps find a source, subjects were first compared by duration of the break time. The thought was that perhaps those who had a shorter break retained more information from the first test as opposed to those who had 2 to 4 hours between testing sessions. The correlations for the 18 subjects who had up to one hour of break and the 18 who had between one to two hours were stronger than that for the overall group with r greater than 0.80 and p less than 0.001. Only the correlation for the 20 who had two to four hours of break was lower, but it was still substantial at 0.652. The scores for each of the groups were not statistically different nor were the change scores significantly different as measured by one-way ANOVA. These results suggest that the different break time durations, while perhaps contributory, do not explain the overall findings.

When evaluated separately, both the control subjects and stroke survivor subjects' r value for overall accuracy was smaller than for the overall group. While both were substantial in magnitude, the stroke survivors' relationship was stronger than the controls (r=0.644 vs r=0.525; p<0.001, single ICC 0.644 vs. 0.481; average ICC 0.781 vs. 0.652). Both findings are to be expected because the two samples have half the subjects. The smaller the sample size, the more likely the values for Pearson r will decrease due to restriction of range. There are fewer scores to compare and inherently less variability—especially in the control group which was anticipated. Interestingly, the difference between the stroke survivors' scores was significant at the 0.01 level but not for the control subjects' scores. Stroke survivors had an average gain of 1.86 points and the controls had an average loss of 0.21 points. The distribution of change scores for the stroke survivors was from −3 to 8 as opposed to −4 to 3 for the controls. This distribution also demonstrates an overall shift towards gain in scores for the stroke survivors and a loss for the control subjects as only 5 stroke survivors dropped in score and 11 improved whereas 12 controls dropped in score and only 10 improved.

The result of comparisons based upon motor ability level for the stroke subjects suggest that what may be indeed learnable is the task of the test: how to explore the objects. The subjects in the stroke survivor group, especially those in the low and medium motor group, had not been asked to perform this kind of matching task prior to this test. Individuals in the low motor ability group had a significant difference between the first and second scores along with the greatest increase in score between the first and second test: 2.63 points. While both the high and medium motor level subjects also had a mean increase in scores, the differences were not found to be significant. While none of the stroke subjects changed in motor ability category between the first two tests, it was apparent on visual observation that the subjects in the low motor group became better at using grasp, lift and release during the second test. This was also supported by the significant drop in average match time (mean=−12.00 seconds, p<0.05) for the low group not demonstrated by the high and medium groups as well as the very strong reliability of the average time to match (r=0.957, p=0.000).

The difference in scores, and the lack of reliability in scores across the three ability groups, may be attributed to improvements based upon learning how to search, as opposed to learning the test objects. One way to test this theory would be to ask the stroke survivors to complete a third test and compare the scores between the second and third test results with the hypothesis that the r values would be greater. Another way to evaluate this theory would be to perform an item by item analysis on the two sets of 18 trials to determine if the person not only improved on these same trials but also on a particular object property category. In addition, the values of the average ICC suggest that averaging two test session scores may be an appropriate indicator of performance in individuals who have low motor ability or other situations where the first experience of the test may not be the most representative indicator of ability. Using the score of 13 as the criterion of impairment, sensitivity for test 2 score is 0.79 and specificity 0.75. If the scores are averaged for the first and second test, sensitivity and specificity are both 0.85 with equivalent values for positive predictive power and negative predictive power of 0.86 and false positive and false negative rates of 0.14. These results lend support for using 13 as the criterion with a zone of uncertainty of 12-14.

Of the three groups of time since injury for the stroke survivors, only the 0-6 month injury group had a significant difference in scores as well as a strong and significant correlation between the two scores. The significant difference could be attributed to the novelty idea, meaning that those who are more recent in injury are less likely to have been asked to perform a match to sample task as opposed to those whose injuries were longer than 6 months before the testing sessions. The strong reliability coefficient r equal to 0.798 and p less than 0.01 may suggest that the results are more reliable for those recently injured, although with a group size of 8, this thought is only speculative. Again, averaging two test scores may be a suitable alternative for these types of individuals. It should be noted, that the restriction of range phenomenon most likely also contributed to these decreases in reliability as discussed previously for the controls subjects.

While the results for the stroke subjects suggest that the test trials are learnable, the results of the control subjects do not support this premise. Why did more of the controls drop in score? Perhaps it can be related to the drop in average match time—the control subjects were more careless in the second test. They may have thought they knew the objects and chose to not explore as much during the second round. Testing subjects in the same day and the desire to finish quickly could also support this phenomenon—as could the boredom theory. Additionally, while the match to sample task was also novel for the control subjects, control subjects have defined and refined search strategies. They did not have to develop those strategies unlike the stroke survivor subjects. If the test trials were indeed learnable, then the majority of subject scores in the entire set should have improved. Regardless of these observations, the 95% confidence interval around the control subject mean scores for the first and second tests overlap with both means fitting within either confidence interval. The confidence interval calculation means that if another subject set of 28 controls were to be tested, there is a 95% certainty that their mean score will fall within that score interval. The first test mean was 14.86 and the 95% confidence interval was 14.86-15.43 and the second test mean was 14.64 with a confidence interval of 13.79-15.49.

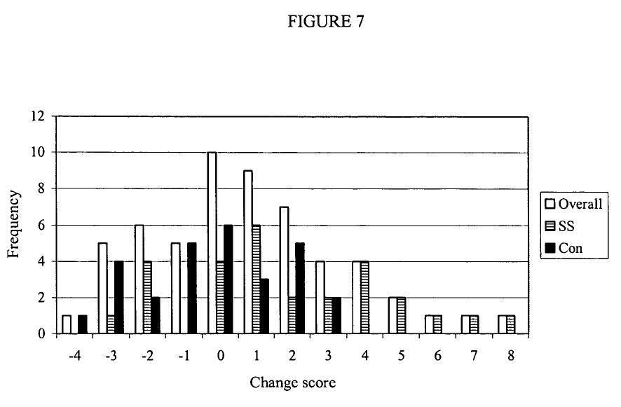

As opposed to the variability in relationships and differences for the two overall accuracy scores, the average time to match scores were consistently very strong. Even though the relationships were very strong, there was a significant difference between the first and second test as everyone matched faster on the second test for most of the comparison's except the high and medium function groups, the 0-1 hour and >1-2 hour break groups, and the greater than 12 months since injury group. The significant change in average match time, if evaluated alone, could mean that the test itself was learnable; however, because the accuracy score did not demonstrate this consistent change the drop in average time match suggests that the subjects simply became more efficient in their explorations. On the other hand, the consistency in relationship supports the theory about average time to match advanced in the validity section. Average time to match, while not necessarily a measure of impairment is more likely a measure of personal preference as reflected in the consistently high values for r between the first and second tests. The lack of correlation between the overall accuracy score and average time to match further supports this theory (see FIG. 7).

1. A device for characterizing the degree of nervous system impairment in a subject, comprising:
a test set having an even number of test objects that are essentially identical in either size or shape, said test set comprising two essentially identical subsets, wherein each of said test objects in a subset varies by one or more increments in at least one of a first and a second parameter, each of which first and second parameters can be assessed by prehension and tactile sensation, and
wherein no more than two test objects in the test set share both the same increment of the first parameter and the same increment of the second parameter.

2. The device according to claim 1, wherein each of said test objects is of sufficient size to fit within and substantially occupy the grasp of a human hand.

3. The device according to claim 1, wherein the test objects are identical in both size and shape.

4. The device according to claim 1, wherein the first and second parameters can be assessed only by prehension and tactile sensation.

5. The device according to claim 1 wherein each of the test objects has a shape that is free of protrusions, voids, and edges.

6. The device according to claim 2 wherein each of the test objects has a shape that is encountered in every day life.

7. The device according to claim 1 wherein the first and second parameters can be discerned by prehension and tactile sensation and directly influence the hand forces that are used to manipulate a test object.

8. The device according to claim 3, wherein the first and second varied parameters are selected from weight, temperature and texture.

9. The device according to claim 3, wherein the first parameter is varied by three increments, and wherein the second parameter is varied by three increments.

10. The device according to claim 9, wherein there are 18 test objects.

11. The device according to claim 10, wherein no more than three test objects in each subset share the same weight, and wherein no more than three test objects in the test set share the same surface texture and the same weight.

12. The device according to claim 1, wherein the size of the test objects is configured to accommodate the body size of the test subject.

13. The device according to claim 11, wherein the varied first and second parameters of the test objects are as set forth in TABLE 1.

14. A method for testing sensory ability in a subject who has sustained injury to the nervous system, comprising;
presenting to the subject in a blinded fashion a combination of test objects from a test set according to claim 1, wherein an examiner and the subject are seated at opposite sides of a table with a screen or curtain disposed between them so as to prevent visual contact, and wherein the combination of test objects is positioned on the same side of the screen or curtain as the examiner and on the opposite side from the subject,
directing the test subject to actively explore by grasping one of the combination of test objects which is identified as a target object, and then to actively explore one or more of the combination of test objects to identify which of the test objects is a match for the target object wherein the test subject's sensory ability is evaluated based on the time taken by the subject to propose a match, and the accuracy of the proposal.

15. The method of claim 14, wherein the test procedure of actively exploring test objects is repeated one or more times, wherein in each trial, a different combination of test objects is used.

16. The method of claim 14, wherein the test procedure of actively exploring test objects is repeated in a series of trials until all possible combinations of objects in the test set of claim 1 have been used.

17. The method of claim 16, wherein the test subject's overall accuracy score is calculated by adding together the number of correct matches, and the test subject's average time to match score is calculated by adding all of the recorded times and dividing by the number of trials.

18. A method for determining the locus of injury to the nervous system in a subject, comprising;
testing the subject according to the method of claim 14,
wherein a low score indicates that an injury is to the somatosensory processing system, and
wherein a low score indicates that an injury is not to the somatosensory processing system.
